(19) United States
(12) Patent Application Publication
(10) Pub. No.: US 2026/0157953 A1
(43) Pub. Date: Jun. 11, 2026

(54) HAIR TREATMENT COMPOSITIONS AND METHODS

(71) Applicant: Conopco, Inc., d/b/a UNILEVER, Hoboken, NJ (US)

(72) Inventors: Nicholas John AINGER, Wageningen (NL); Robert MACHEN, Wageningen (NL); Aneliya Nikolova ZDRAVKOVA, Wageningen (NL)

(21) Appl. No.: 19/122,652

(22) PCT Filed: Oct. 26, 2023
(86) PCT No.: PCT/EP2023/079933

§ 371 Date: Apr. 18, 2025

(30) Foreign Application Priority Data
Nov. 9, 2022 (EP) 22206273.9

(51) Int. Cl. A61K 8/81; A61Q 5/00; A61Q 5/02; A61Q 5/12

(52) U.S. Cl. CPC A61K 8/817; A61Q 5/006; A61Q 5/02; A61Q 5/12; A61K 2800/30; A61K 2800/5424; A61K 2800/5426; A61K 2800/5428

(57) ABSTRACT

A sulphate-free cleansing composition for hair and scalp comprising: i) of an alpha olefin sulfonate anionic surfactant of general formula (I): R¹—CH═CH—CH₂—SO₃-M⁺ (I) in which R¹ is selected from linear or branched alkyl groups having from 11 to 13 carbon atoms and mixtures thereof; and M is a solubilizing cation; (ii) an amphoteric or zwitterionic surfactant, selected from an alkyl betaine of general formula (II) R²—N⁺(CH₃)₂—CH₂—COO-M⁺ (II) wherein R²═C12 (Lauryl), Cocamidopropyl or Coco derived; an alkyl hydroxy sultaine of general formula (III), R³—N⁺(CH₃)₂—CH₂—CH(OH)—CH₂—SO₃-M⁺ (III) wherein R³=Cl₂ (Lauryl) or Coco derived; an alkyl aminopropyl hydroxy sultaine of general formula (IV), R⁴—CO—NH—(CH₂)₃—N⁺(CH₃)₂—CH₂—CH(OH)—CH₂—SO₃-M⁺ (IV) wherein R⁴=C12 (Lauryl) or Coco derived; an alkyl amphoacetate of general formula (V), R⁵—CO—NH—(CH₂)₂—N(CH₂—CH₂—OH) (CH₂—COO-M⁺) (V) wherein R⁵=C12 (Lauryl) or Coco derived; and mixtures thereof and; iii) a piroctone compound; wherein the weight ratio of alpha olefin sulfonate anionic surfactant (i) to amphoteric or zwitterionic surfactant (ii) ranges from 1:1 to 6:1.

### FILED OF THE INVENTION

The present invention relates to a hair treatment composition, particularly an anti-dandruff shampoo composition.

### BACKGROUND OF THE INVENTION

Dandruff is a problem affecting many people globally. The condition is manifested by the shedding of clumps of dead skin cells from the scalp, these are white in colour and provide an aesthetically displeasing appearance. A factor that contributes to dandruff are certain members of the Malassezia yeasts. To combat these yeasts, hair treatment compositions are developed including various actives for their antidandruff effectiveness. Piroctone compound such as piroctone olamine is one such active.

A common problem with piroctone compound is that deposition of the active onto the desired surface during wash process is minimum. The desired surface is typically scalp and/or hair. For example, piroctone compound such as piroctone olamine is usually soluble in surfactants of the cleansing phase comprised in a hair treatment composition. During the excessive rinsing process, the majority of piroctone is likely to be washed away together with the surfactants. Poor deposition is correlated with low antidandruff activity, thus little mitigation of the ill-effects of dandruff. To date, there are attempts to offset this drawback by increasing the level of piroctone olamine in hair treatment composition. Such approach causes a variety of issues such as increased costs, potential instability of the formulation and potential adverse effect to hair sensory. Hence it is not an approach favoured by the industry.

DE102012203240A1 describes hair treatment composition with increased antidandruff effect and improved combing, gloss, elasticity of hair. The composition includes antidandruff agent selected from zinc pyrithione, climbazole, octopirox, ketoconazol, selenium disulphide, selenium containing vegetable oil, selenium containing plant extract, and a cationic amino silicone. Examples include shampoos and conditioners comprising Octopirox® (Piroctone Olamine), amino silicone and surfactants.

US2013/0059929A1 describes a cosmetic or dermatological preparation, wherein the preparation comprises at least one of benzethonium chloride, methylisothiazolinone, piroctone olamine, and lauroyl ethyl arginate, and does not contain a preservative that comprises a phenolic group. The preparations show improved sensory properties and sufficient microbiological stability.

US2002/0035161A1 describes a cosmetic/pharmaceutical oil-in-water emulsion including a discontinuous fatty phase dispersed in a continuous aqueous phase and comprising an effective amount of at least one biologically active agent (A) and an effect amount of an emulsifying system (B) therefore, said at least one biologically active (A) being non-solubilized therein in micronized particulate state, at least 80%, numerically, of said micronized particles having diameters ranging from 1 to 10 μm and at least 50%, also numerically, having diameters of less than 5 μm.

Despite all the prior art, there remains a need to improve the deposition of piroctone compounds, especially piroctone acid or piroctone olamine, onto the surface of scalp and/or hair during washing process. There also remains a further need to improve the deposition onto said surface without adverse side effects to sensory, formulation rheology and conditioning performance.

### DESCRIPTION OF THE INVENTION

The present invention relates to a sulphate-free cleansing composition for hair and scalp comprising:
i) of an alpha olefin sulfonate anionic surfactant of general formula (I):

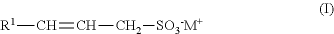

in which R¹ is selected from linear or branched alkyl groups having from 11 to 13 carbon atoms and mixtures thereof; and M is a solubilizing cation;
(ii) an amphoteric or zwitterionic surfactant, selected from an alkyl betaine of general formula (II)

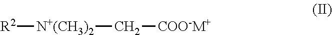

wherein R²=C12 (Lauryl), Cocamidopropyl or Coco derived;
an alkyl hydroxy sultaine of general formula (III),

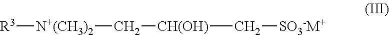

wherein R³=C12 (Lauryl) or Coco derived;
an alkyl aminopropyl hydroxy sultaine of general formula (IV),

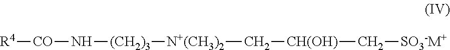

wherein R⁴=C12 (Lauryl) or Coco derived;
an alkyl amphoacetate of general formula (V),

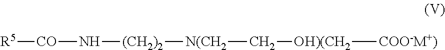

wherein R⁵=C12 (Lauryl) or Coco derived;
and mixtures thereof and;
iii) a piroctone compound;
wherein the weight ratio of alpha olefin sulfonate anionic surfactant (i) to amphoteric or zwitterionic surfactant (ii) ranges from 1:1 to 6:1.

The invention also relates to a non-therapeutic method of treating hair or the scalp, comprising application of the personal care composition described above.

Further described is a composition as above for use in the treatment of the hair or scalp.

For the avoidance of doubt, any feature of one aspect of the present invention may be utilised in any other aspect of the invention. Any feature described as ‘preferred’ should be understood to be particularly preferred in combination with a further preferred feature or features. Herein, any feature of a particular embodiment may be utilised in any other embodiment of the invention. The examples given in the description below are intended to clarify the invention but not to limit the invention. All percentages are weight/weight percentages unless otherwise indicated.

### DETAILED DESCRIPTION OF THE INVENTION

By “aqueous continuous phase” it is meant a continuous phase which has water as its basis.

In the context of the present invention sulphate free means comprising less than 0.1 wt % of the total composition of a sulphate surfactant, preferably less than 0.05 wt %.

Suitably, the composition of the invention will comprise from about 60 to about 95%, preferably from 65 to 95%, more preferably from 70 to 90% water (by weight based on the total weight of the composition).

Preferably the composition comprises an isotropic surfactant phase, where under dilution, isotropic micelles provide higher availability of monomers to the air/water interface, whereas anisotropic may diffuse at a slower rate, resulting in lower flash foam properties. Thus, the isotropic phase is advantageous for product appearance, clarity and good flash foam properties.

All amounts referred to herein are based on 100% activity (or “active”) unless otherwise stated. By 100% activity (or “active”) is meant that the material is not diluted and is at 100% v/v or wt/wt. Many materials used in personal care formulations are commercially available at different active concentrations, for example at 70% active or 60% active. For example, 100 ml of 70% active surfactant provides the same amount of active material as 70 ml of 100% active surfactant. Therefore, in order to provide for variations in activities of materials, all amounts are based on 100% active materials.

The aqueous continuous phase comprises a total amount of anionic, amphoteric and zwitterionic surfactant consisting of (i) and (ii) below. That is to say, no further anionic, amphoteric and zwitterionic surfactants are present in the compositions of the invention. Preferably, no other surfactants, for example, nonionic surfactants are present in the compositions of the invention.

### (i) the Alpha Olefin Sulfonate Anionic Surfactant

The composition of the invention comprises (i) one or more alpha olefin sulfonate anionic surfactants of general formula (I)

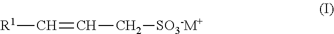

in which R¹ is selected from linear or branched alkyl groups having from 11 to 13 carbon atoms and mixtures thereof; and M is a solubilizing cation;

Preferably R¹ in general formula (I) is a C₁₄ or C₁₆ linear alkyl group. Preferably R¹ in general formula (I) is a C₁₁ or C₁₃ linear alkyl group.

Preferably M in general formula (I) is selected from alkali metal cations (such as sodium or potassium), ammonium cations and substituted ammonium cations (such as alkylammonium, alkanolammonium or glucammonium).

Commercially produced alpha olefin sulfonate anionic surfactants of general formula (I) may be made by sulfating C14-16 olefins derived from natural gas. The process can also yield mixtures of homologues and low levels of unreacted olefins.

Particularly preferred is alpha olefin sulfonate with an average of 14-16 carbons. A suitable example of such a material is Bioterge AS40 (ex Stepan).

The amount of alpha olefin sulfonate anionic surfactant, at 100% activity, of general formula (I) ranges from 3 to 13%, preferably from 3.5 to 12%, more preferably from 3 to 10%, (by weight based on the total weight of the composition).

### (ii) The Amophoteric or Zwitterionic Surfactant of General Formulae (II), (III), (IV) or (V)

The composition of the invention comprises (ii) an amphoteric or zwitterionic surfactant, selected from an alkyl betaine of general formula (II)

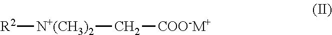

wherein R²=C12 (Lauryl), Cocamidopropyl or Coco derived;
an alkyl hydroxy sultaine of general formula (III),

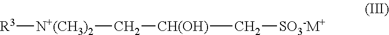

wherein R³=C12 (Lauryl) or Coco derived;
an alkyl aminopropyl hydroxy sultaine of general formula (IV),

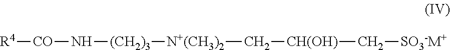

wherein R⁴=C12 (Lauryl) or Coco derived;
an alkyl amphoacetate of general formula (V),

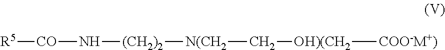

wherein R⁵=C12 (Lauryl) or Coco derived;
and mixtures thereof.

Preferably, R²=C12 (Lauryl) or Coco derived.

The preferred surfactant (ii) is selected from coco betaine, cocamidopropyl betaine, lauryl hydroxy sultaine, coco aminopropyl hydroxy sultaine, lauryl amphoacetate and mixtures thereof, most preferably selected from coco betaine, cocamidopropyl betaine, lauryl hydroxy sultaine and mixtures thereof. Alkyl hydroxy sultaines are preferred, particularly lauryl hydroxy sultaine.

The amount of amphoteric or zwitterionic surfactants of general formula (II), (III), (IV) or (V) or mixtures thereof, preferably ranges from 1 to 6%, more preferably from 1.5 to 5%, most preferably from 2 to 3.5% (based on the total weight of the composition and 100% activity).

In a more preferred composition the amphoteric or zwitterionic surfactant (ii) is selected from a betaine amphoteric surfactant of general formula (II), which is coco betaine or cocamidopropyl betaine, an amphoteric surfactant of general formula (III), which is lauryl hydroxy sultaine, and mixtures thereof, in an amount of from 1 to 4% (by weight based on the total weight of the composition and 100% activity).

The combined amount of (i) and (ii) preferably ranges from 4 to 20 wt %, preferably from 5 to 17 wt %, most preferably from 8 to 15 wt % (based on the total weight of the composition and 100% activity).

An especially preferred composition according to the invention comprises (i) alpha olefin sulfonate in an amount ranging from 3.25 to 8% (by weight based on the total weight of the composition and 100% active material); and (ii) an amphoteric or zwitterionic surfactant selected from coco betaine, cocamidopropyl betaine, lauryl hydroxy sultaine, coco aminopropyl hydroxy sultaine, lauryl amphoacetate or mixtures thereof, in an amount ranging from 1 to 4% (by weight based on the total weight of the composition and 100% active material).

The weight ratio of the alpha olefin sulfonate anionic surfactant (i) to the amphoteric surfactant (ii) ranges from 1:1 to 6:1, preferably from 1.5:1 to 5.

Preferably, the pH of the composition of the invention ranges from 3 to 6.5, preferably from 3.5 to 5.1, more preferably from 4 to 5.

A protonating agent may be used for achieving the low pH. Suitable protonating agents are acids. Suitable acids useful herein include hydrochloric acid, citric acid, acetic acid, tartaric acid, fumaric acid, lactic acid, malic acid, succinic acid, and mixtures thereof. Preferably, the acid is selected from the group consisting of citric acid, acetic acid, tartaric acid, hydrochloric acid, fumaric acid, lactic acid and mixtures thereof.

The piroctone compound for use in the present invention include piroctone acid, primary, secondary and tertiary olamine salts of piroctone acid (such as the diethanolamine and triethanolamine salts), and mixtures thereof, preferably piroctone acid, primary olamine salt of piroctone acid (i.e. piroctone olamine, also known as Octopirox®) and mixtures thereof.

Piroctone Olamine is an olamine salt of the hydroxamic acid derivative piroctone. It is commonly known as piroctone ethanolamine with the trade name Octopirox®.

The piroctone olamine according to the present invention is a 1:1 compound of 1-hydroxy-4-methyl-6-(2,4,4-trimethylpentyl)-2(1H)-pyridinone with 2-aminoethanol and is also designated 1-hydroxy-4-methyl-6-(2,4,4-trimethylpentyl)-2(1H) pyridinone monoethanolamine salt. The CAS number is 68890-66-4 and the compound has the general formula (I) as below:

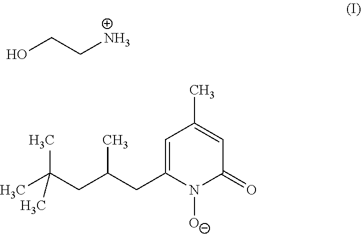

The piroctone compound, particularly the piroctone olamine is preferably present from 0.01 to 5% by weight of the total composition, more preferably from 0.1 to 5 wt %, most preferably from 0.2 to 3 wt %, of the total composition.

The personal care composition may comprise at least one cationic deposition polymer.

The cationic polymer comprises a dimethyl diallyl ammonium moiety.

Preferred cationic deposition polymers are homo or copolymers where a dimethyl diallyl ammonium moiety is one of the monomers. More preferred cationic polymers are polyquaternium-6, polyquaternium-7 and polyquaternium-22. A particularly preferred cationic polymer is polyquaternium-6.

Typically, the cationic polymer is present at a level of from 0.01 to 5% by weight of the total composition, preferably from 0.05 to 2 wt %, more preferably from 0.1 to 1 wt %.

Additional cationic deposition polymers may be included, but they are not preferred.

Other cationic polymers include polygalactomannans and polysaccharide polymers, such as cationic cellulose derivatives and cationic starch derivatives.

Mixtures of any of the above cationic polymers may be used.

Preferably the weight ratio of cationic polymer to total surfactant, preferably the olefin sulfonate anionic surfactant i( ) and amphoteric or zwitterionic surfactant (ii) is from 20:1 to 50:1, preferably from 25:1 to 40:1.

Preferably the composition of the invention, particularly an aqueous shampoo composition for use in the invention further comprises a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich.

Suspending agent will generally be present in a shampoo composition for use in the invention at levels of from 0.1 to 10%, preferably from 0.15 to 6%, more preferably from 0.2 to 4% by total weight of suspending agent based on the total weight of the composition.

The cosmetic composition may optionally comprise one or more components, provided that the optional components are physically and chemically compatible with the essential components described hereinbefore, and do not otherwise unduly impair sensory, formulation rheology and conditioning performance. Individual concentrations of such optional components may range from 0.001% to 10% by weight of the total composition, preferably from 0.01% to 5% wt %. Such components may include conditioning agents, fragrance, dyes, pigments, pH adjusting agents, pearlescers or opacifiers, viscosity modifiers, preservatives, and natural hair nutrients such as botanicals, fruit extracts, sugar derivatives and amino acids.

One of the preferred optional components is a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich. Suitable cross-linked polymers of acrylic acid and acrylate esters are Pemulen TR1 or Pemulen TR2. A suitable heteropolysaccharide gum is xanthan gum, for example that available as Kelzan mu.

Mixtures of any of the above suspending agents may be used. Preferred is a mixture of cross-linked polymer of acrylic acid and crystalline long chain acyl derivative.

A most preferred example is a crosslinked polyacrylate polymer.

Suspending agent, if included, will generally be present in the composition at a level of from 0.01 to 5 wt. %, preferably from 0.1 to 2.5 wt. %, more preferably from 0.25 to 1 wt. %.

Another preferred optional component is a conditioning agent, providing conditioning benefit. Typically, the most popular conditioning agents used in cosmetic compositions are water-insoluble oily materials such as mineral oils, naturally occurring oils such as triglycerides and silicone oils. Conditioning benefit is achieved by the oily material being deposited onto the hair resulting in the formation of a film, which makes the hair more lubricated and less dry. Preferably, the conditioning agent is non-volatile, meaning that it has a vapour pressure of less than 1000 Pa at 25° C.

Preferably, the composition comprises discrete dispersed droplets of a water-insoluble conditioning agent, which has a mean droplet diameter (D_{3,2}) of less than 50 microns, preferably less than 30 microns, more preferably less than 15 microns, most preferably less than 10 microns. The mean droplet diameter (D_{3,2}) of a water-insoluble conditioning agent may be measured by means of a laser light scattering technique, for example using a 2600D Particle Sizer from Malvern Instruments.

The water-insoluble conditioning agent may include non-silicone conditioning agent comprising non-silicone oily or fatty materials such as hydrocarbon oils, fatty esters and mixtures thereof. Preferably, the water-insoluble conditioning agent is emulsified silicone oil.

Suitable silicones include polydiorganosiloxanes, in particular polydimethylsiloxanes which have the CTFA designation dimethicone. Also suitable for use in compositions of this invention (particularly shampoos and conditioners) are polydimethyl siloxanes having hydroxyl end groups, which have the CTFA designation dimethiconol. Also suitable for use in compositions of this invention are silicone gums having a slight degree of cross-linking, as are described for example in WO 96/31188. Preferably, the silicone oil comprises dimethicone, dimethiconol or a mixture thereof.

The viscosity of the emulsified silicone itself (not the emulsion or the final hair care composition) is typically at least 10,000 cSt (centi-Stokes=mm²·S⁻¹) at 25° C., preferably at least 60,000 cSt, most preferably at least 500,000 cSt, ideally at least 1,000,000 cSt. Preferably the viscosity does not exceed 10° cSt for ease of formulation. Suitable methods for measuring the kinematic viscosity of silicone oils are known to those skilled in the art, e.g. capillary viscometers. For high viscosity silicones, a constant stress rheometer can be used to measure viscosity.

Suitable emulsified silicones for use in the compositions are available as pre-formed silicone emulsions from suppliers of silicones such as Dow Corning and GE silicones. The use of such pre-formed silicone emulsion is preferred for ease of processing and control of silicone particle size. Such pre-formed silicone emulsions will typically additionally comprise a suitable emulsifier and may be prepared by a chemical emulsification process such as emulsion polymerisation, or by mechanical emulsification using a high shear mixer.

Examples of suitable pre-formed silicone emulsions include DC1785, DC1788, DC7128, all available from Dow Corning. These are emulsions of dimethiconol/dimethicone.

Another class of silicones which may be used are functionalized silicones such as amino functional silicones, meaning a silicone containing at least one primary, secondary or tertiary amine group, or a quaternary ammonium group. Examples of suitable amino functional silicones include polysiloxanes having the CTFA designation “amodimethicone.”

Preferably, silicone emulsion droplets are blended with certain types of surface active block polymers of a high molecular weight to form silicone emulsions, as described for example in WO03/094874. One preferred form of the surface active block polymer having polyoxypropylene and polyoxyethylene groups as the hydrophobic and hydrophilic part respectively has formula V and has the CTFA designation poloxamer, known commercially under the trade name “Pluronic” from BASF.

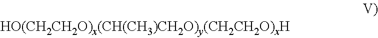

Suitably, the mean value of x in formula I is 4 or more, preferably 8 or more, more preferably 25 or more, yet more preferably 50 or more and most preferably 80 or more. The mean value of x is typically no greater than 200. Suitably, the mean value of y is 25 or more, preferably 35 or more, more preferably 45 or more and most preferably 60 or more. The mean value of y is typically no greater than 100.

Another preferred form of the surface active block polymer is according to formula VI and has the CTFA designation Poloxamine. Those are commercially available under the trade name “Tetronic” from BASF.

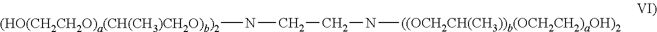

Suitably, the mean value of a is 2 or more, preferably 4 or more, more preferably 8 or more, even more preferably 25 or more and most preferably 40 or more. The mean value of a is typically no greater than 200. The mean value of b is suitably 6 or more, preferably 9 or more, more preferably 11 or more and most preferably 15 or more. The mean value of b is typically no greater than 50.

Preferably, the surface active block polymer is poloxamer and/or poloxamine, more preferably, the surface active block polymer is poloxamer.

Preferably, the surface active block polymer is blended with dimethicone. The weight ratio of dimethicone to surface active block polymer in the blend is preferably in the range from 2:1 to 200:1, more preferably from 5:1 to 50:1, even more preferably from 10:1 to 40:1, most preferably from 15:1 to 30:1.

The water-insoluble conditioning agent is generally present in the composition in an amount from 0.05 to 15%, preferably from 0.1 to 10%, more preferably from 0.5 to 8%, most preferably from 1 to 5%, based on the total weight of the composition and including all ranges subsumed therein.

The composition may preferably comprise a pearlescer. The preferred pearlescer is ethylene glycol ester as disclosed in U.S. Pat. No. 4,885,107, incorporated herein by reference. Preferably, the ethylene glycol ester is a mono- or, di-ester of glycol, more preferably a di-ester of glycol.

Preferably ethylene glycol mono- or di-ester is an ethylene glycol mono- or di-ester of a C₁₂₋₂₂ fatty acid, more preferably an ethylene glycol mono- or di-ester of a saturated C₁₂₋₂₂ fatty acid. Most preferred are the diesters comprising a mixture of palmitate and stearate. The amount of stearate should be in the range of about 10% to about 42% or in the range of about 55% to about 80% with palmitate accounting for the remainder. The amount of stearate is preferably from about 60% to about 75%, more preferably from about 80-95%, most preferably 100%. The most suitable example of a pearlescer is an ethylene glycol distearate. Pearlescer may also perform the function as a suspending agent.

The level of the ethylene glycol ester can be suitably from about 0.5% to about 6%, preferably from about 1% to about 4%, by weight of the total composition.

The viscosity of the composition suitably ranges from 3,000 to 10,000 mPa·s, preferably from 4,000 to 8,000 mPa·s, more preferably from 5,000 to 7,000 mPa·s when measured using a Brookfield V2 viscometer (spindle RTV5, 1 minute, 20 rpm) at 30° C.

Rinse-off compositions are intended to be rinsed off the scalp of the user with water after use. Rinse off compositions are preferred. Leave-on composition is intended not to be rinsed off the scalp of the user immediately after use (i.e. within at least the first 2 hours, preferably at least 4 hours after application of the composition). In the context of the present invention rinse-off compositions include shampoos and conditioners, as well as treatment compositions which can be left on scalp for from 0.5 minute to up to 15 minutes, preferably from 1 minute to 10 minutes, more preferably 1 minutes to 10 minutes, prior to being rinsed off.

The preferred use of the cosmetic composition is in a shampoo, particularly a rinse off shampoo. Shampoo compositions for use in the invention are generally aqueous, i.e. they have water or an aqueous solution or a lyotropic liquid crystalline phase as their major component. Suitably, the shampoo composition will comprise from 50 to 98%, preferably from 60 to 92% water by weight based on the total weight of the composition. Such compositions are referred to as having an aqueous base.

The invention will now be illustrated by the following non-limiting Examples

### Examples

Compositions were made according to Table 1 and 2.

TABLE 1

| Material | A | 1 | 2 | 3 | B | 4 | 5 | 6 |
|---|---|---|---|---|---|---|---|---|
| Sodium Laureth Sulphate | 13 | | | | 13 | | | |
| Cocoamidopropyl Betaine | 1.5 | | | | 1.5 | | | |
| Alpha Olefin Sulfonate | | 9 | 9.75 | 10.9 | | 9 | 9.75 | 10.9 |
| Lauryl Hydroxy Sultaine | | 3 | 3.25 | 3.6 | | 3 | 3.25 | 3.6 |
| Dimethiconol/TEA -dodecylbenzene Sulfonate | 2 | 2 | 2 | 2 | 2 | 2 | 2 | 2 |
| Carbomer | 0.4 | 0.4 | 0.4 | 0.4 | 0.4 | 0.4 | 0.4 | 0.4 |
| Perfume | 0.8 | 0.8 | 0.8 | 0.8 | 0.8 | 0.8 | 0.8 | 0.8 |
| Piroctone olamine | 0.5 | 0.5 | 0.5 | 0.5 | 0.5 | 0.5 | 0.5 | 0.5 |
| Water & Minors (preservative, citric & salt) | to 100 | | | | | | | |
| Polyquaternium-6 (PDADMAC) | 0.35 | | | | 0.50 | | | |
| Deposition/ppm | 6.53 | 13.0 | 18.3 | 9.53 | 7.42 | 14.1 | 10.5 | 16.3 |
| Standard error | 0.76 | 1.3 | 1.79 | 1.6 | 0.94 | 0.4 | 0.98 | 0.80 |
| Percentage change in deposition from control/ | 0 | 99.1 | 180.2 | 45.9 | 0 | 90.0 | 41.5 | 119.7 |
| % | | | | | | | | |

TABLE 2

| Material | C | 7 | 8 | D | 9 |
|---|---|---|---|---|---|
| Sodium Laureth Sulphate | 13 | | | 13 | |
| Cocoamidopropyl Betaine | 1.5 | 3.7 | 4.8 | 1.5 | 4.8 |
| Alpha Olefin Sulfonate | | 10.9 | 7.2 | | 7.2 |
| Lauryl Hydroxy Sultaine | | | | | |
| Dimethiconol/TEA -dodecylbenzene | 2 | 2 | 2 | 2 | 2 |
| Sulfonate | | | | | |
| Carbomer | 0.4 | 0.4 | 0.4 | 0.4 | 0.4 |
| Perfume | 0.8 | 0.8 | 0.8 | 0.8 | 0.8 |
| Piroctone olamine | 0.5 | 0.5 | 0.5 | 0.5 | 0.5 |
| Water & Minors (preservative, | to 100 | | | | |
| citric & salt) | | | | | |
| Polyquaternium -6 (PDADMAC) | 0.35 | | | 0.50 | |
| Percentage change in deposition | 0 | 54.7 | 58.5 | 0 | 32.9 |
| from control/% | | | | | |

The deposition of the piroctone olamine was measured by thoroughly wetting with water 10 clean, dark-brown European hair switches (2.5 g/6″). 5 replicas were tested for each shampoo composition. 0.25 g shampoo was applied to one switch followed by 30 second massage. The switch was then rinsed by water for 30 seconds. 0.25 g shampoo was re-applied followed by another 30 second massage. The shampoo was then rinsed off by water for 30 seconds. The switch was allowed to dry naturally. The dried switch was extracted in 50 ml ethanol. Caution was taken not to expose the extraction to UV light. The extraction was analysed by UPLC for piroctone acid deposition. The results are shown above in Tables 1 and 2.

The results demonstrate that the Example of the invention is more effective at depositing Piroctone Olamine than the comparative Examples.

1. A sulphate-free cleansing composition for hair and scalp comprising:
i) of an alpha olefin sulfonate anionic surfactant of general formula (I): 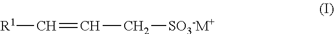
in which R¹ is selected from linear or branched alkyl groups having from 11 to 13 carbon atoms and mixtures thereof; and M is a solubilizing cation;
(ii) an amphoteric or zwitterionic surfactant, selected from an alkyl betaine of general formula (II) 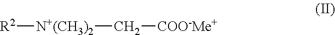
wherein R²=C12 (Lauryl), Cocamidopropyl or Coco derived;
an alkyl hydroxy sultaine of general formula (III), 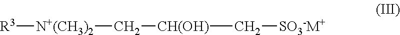
wherein R³=C12 (Lauryl) or Coco derived;
an alkyl aminopropyl hydroxy sultaine of general formula (IV), 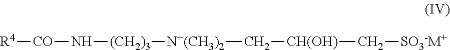
wherein R⁴=C12 (Lauryl) or Coco derived;
an alkyl amphoacetate of general formula (V), 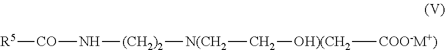
wherein R⁵=C12 (Lauryl) or Coco derived;
and mixtures thereof; and
iii) a piroctone compound;
wherein the weight ratio of alpha olefin sulfonate anionic surfactant i) to amphoteric or zwitterionic surfactant ii) ranges from 1.5:1 to 5:1.

2. The sulphate free cleansing composition according to claim 1, wherein piroctone compound is piroctone olamine.

3. The sulphate free cleansing composition according to claim 1, wherein the piroctone compound is present in an amount from 0.1 to 5 wt % of the total composition.

4. The sulphate free cleansing composition according to claim 1, further comprising a cationic deposition polymer.

5. (canceled)

6. The sulphate free cleansing composition according to claim 4, wherein the weight ratio of cationic polymer to total surfactant is from 20:1 to 50:1.

7. The sulphate free cleansing composition according to claim 1, wherein the composition has a pH from 3 to 6.5.
